(19) United States
(12) Patent Application Publication
(10) Pub. No.: US 2013/0204138 A1
(43) Pub. Date: Aug. 8, 2013

(54) STEERABLE CATHETER NAVIGATION WITH THE USE OF INTERFERENCE ULTRASONOGRAPHY

(71) Applicants: Marek Belohlavek, Scottsdale, AZ (US); Eileen M. McMahon, Mesa, AZ (US)

(72) Inventors: Marek Belohlavek, Scottsdale, AZ (US); Eileen M. McMahon, Mesa, AZ (US)

(21) Appl. No.: 13/816,796

(22) PCT Filed: Aug. 15, 2011
(86) PCT No.: PCT/US11/47711

§ 371 Date: Apr. 24, 2013

Related U.S. Application Data
(60) Provisional application No. 61/375,093, filed on Aug. 19, 2010.

(51) Int. Cl. A61B 8/00; A61B 8/12; A61B 8/08

(52) U.S. Cl. CPC A61B 8/4254; A61B 8/0891; A61B 8/12; A61B 8/445; A61B 8/488; A61B 8/54; A61B 8/4483; A61B 8/5207; A61B 8/461
USPC 600/453; 600/463

(57) ABSTRACT

An acoustically-active catheter (AAC) for use with an ultrasound imaging system and a method for tracking the AAC with respect to anatomic target chosen within a body using the interference ultrasonography. A tip of the AAC is equipped with a crystal transmitting an acoustic wave with parameters similar to those of an acoustic wave generated by a transducer of the imaging system so as to produce acoustic interference the strength of which depends on a position of the AAC tip with respect to at least one Doppler scan plane, formed by the imaging transducer, and a distance from a pulsed-wave Doppler sample that is overlapped with the anatomic target. The ultrasound imaging system detects the interference signal and produces a visual and/or audible interference output that indicates the strength of the acoustic interference. Based on the intensity of the interference output, the user can navigate the AAC tip towards the anatomic target. An anatomic target can be chosen within a cardiovascular system.

### CROSS-REFERENCE TO RELATED APPLICATIONS

The present application claims benefit of and priority from the U.S. Provisional Patent Application No. 61/375,093 filed on Aug. 19, 2010 and titled “Steerable Catheter Navigation With the Use of Interference Ultrasonography.” The disclosure of the above-mentioned application is incorporated herein by reference in its entirety.

### STATEMENT REGARDING FEDERALLY SPONSORED RESEARCH OR DEVELOPMENT

This invention was made with government support under Grant No. EB009111 awarded by the National Institute of Health. The government has certain rights in this invention.

### TECHNICAL FIELD

The present invention relates to ultrasonic imaging systems and methods and, more particularly, to ultrasonic systems and methods capable of locating a position of an object, such as a catheter, within a body by utilizing acoustic interference between an acoustic wave generated by the object and the ultrasound (US) wave generated by the imaging system.

### BACKGROUND ART

Various ways are known in which ultrasound can be used to produce images of objects. In the so-called transmission imaging, for example, an ultrasound transmitter may be placed on one side of an object so as to have sound transmitted through the object to the ultrasound receiver that is placed on the other side of the object. With the transmission method, an image may be produced in which brightness of each pixel of an image is a function of the amplitude of the ultrasound that reaches the receiver (“attenuation” mode), or the brightness of each pixel of the displayed image is a function of the time required for the sound to reach the receiver (“time-of-flight” or “speed of sound” mode). In the alternative, the receiver may be positioned on the same side of the object as the transmitter and an image may be produced in which the brightness of each pixel is a function of the amplitude or time-of-flight of the ultrasound reflected from the object back to the receiver (this is referred to as “reflection”, “backscatter” or “echo” imaging).

Several backscatter methods for acquiring ultrasound data are known. In the so-called “A-scan” method, an ultrasound pulse is directed into the object by the transducer and the amplitude of the reflected sound is recorded over a period of time. The amplitude of an echo signal is proportional to the scattering strength of reflecting elements in the object and the time delay is proportional to a distance separating these reflectors from the transducer. In the so-called “B-scan” method, the transducer transmits a series of ultrasonic pulses as it is scanned across the object along a single axis of motion. The resulting echo signals are recorded in a fashion similar to that of the A-scan method and their amplitudes are used to modulate the brightness of pixels on a display. The location of the transducer and the time delay values of the received echo signals determine the display pixels to be illuminated. With the B-scan method, enough data are acquired from which a two-dimensional image of the reflecting elements can be reconstructed. Rather than physically moving the transducer over the subject to perform a scan, sometimes an array of transducer elements is employed while an ultrasonic beam is electronically moved or scanned (swapped) over a region of interest.

Ultrasonic transducers for medical applications are known to include one or more piezoelectric elements sandwiched between a pair of electrodes. A typical piezoelectric element is constructed of lead zirconate titanate (PZT), polyvinylidene diflouride (PVDF), or PZT ceramic/polymer composite. The electrodes of the piezo-element are connected to a voltage source, and application of voltage to the piezo-element triggers its change of dimensions at a frequency corresponding to that of the applied voltage. When a voltage pulse is applied, the piezoelectric element emits an ultrasonic wave, into the media to which it is coupled, at frequencies present in the excitation pulse. Conversely, when an ultrasonic wave strikes the piezoelectric element, the element produces a corresponding voltage across its electrodes. Typically, the front of the element is covered with an acoustic matching layer that improves acoustical coupling with the media in which the ultrasonic waves propagate. In addition, a backing material may be disposed to the rear of the piezoelectric element to absorb ultrasonic waves that emerge from the back side of the element so that they do not interfere. A number of such ultrasonic transducer constructions have been disclosed (see, e.g., U.S. Pat. Nos. 4,217,684; 4,425,525; 4,441,503; 4,470,305 and 4,569,231).

When used for ultrasound imaging, the transducer typically has a number of piezoelectric elements arranged in an array and driven with separate voltages (apodizing). By controlling the time delay (or phase) and amplitude of the applied voltage signals, the ultrasonic waves produced by such a phase array of piezoelectric elements (in the transmission mode) combine to create a net ultrasonic wave focused at a selected point. By controlling the time delay and amplitude of the applied voltages, this focal point can be moved in a plane to scan the subject.

The same principles apply when the transducer is employed to receive the object sound (using echo imaging approach). Specifically, the voltage signals produced at the transducer elements in a phase-array are summed together such that the net signal is indicative of the sound reflected from a single focal point in the object. As with the transmission imaging, this focused reception of the ultrasonic energy is achieved by imparting separate time delay (and/or phase shifts) and gains to the echo signal received by each transducer array element.

Doppler Imaging.

Doppler systems employ ultrasonic pulses (pulsed-wave, or PW, Doppler) or continuous acoustic beam (continuous-wave, or CW, Doppler) to measure the velocity of moving reflectors, such as flowing blood cells (“flow” Doppler) or moving cardiac walls (“tissue” Doppler). Velocity is detected by measuring the Doppler shifts in frequency imparted to the ultrasound signal from the moving reflectors. The PW Doppler method is suitable for defining a small sample volume within which velocity of reflectors is measured, whereas the CW Doppler method is typically preferred for measurement of maximum velocity of reflectors moving along the ultrasound beam.

Doppler imaging may be incorporated in a real-time scanning imaging system. The system provides electronic steering and focusing of a single acoustic beam and enables small volumes to be illuminated anywhere in the field of view of the instrument, whose locations can be visually identified on a two-dimensional B-scan image. A Fourier transform processor faithfully computes the Doppler spectrum backscattered from the sampled volumes, and by averaging the spectral components the mean frequency shift can be obtained. Typically the calculated velocity is expressed in the B-scan image by color-coding individual pixels.

Catheter Guidance.

With the advent of numerous minimally invasive procedures, proper catheter guidance is becoming increasingly important. Within the field of interventional medicine, catheters have become widely used for a number of both diagnostic and therapeutic procedures. Fore example, in the particular field of cardiology, catheters and catheter-based tools are used for coronary angiograms, cardiac ablation, and percutaneous procedures including coronary interventions including angioplasty, atherectomy, and stent or closure device placement. Other medical specialties also use catheters for various purposes including fluid drainage, injections, and biopsy or so-called minimally-invasive surgical procedures. Future application of catheters may also include precise in situ delivery of personally tailored drugs or gene therapy.

Regardless of US equipment used and the type of scan employed by this equipment, the interventions into the cardiovascular system of a patient by the imaging-navigation system have to be minimally invasive in order to be advantageous in comparison with the full-extent (open-chest) surgery and direct (visual) navigation of tools and instruments by a surgeon or skilled and qualified operator.

The related art describes cardiovascular catheter navigation. Traditionally, cardiac catheterization procedures have been done under the guidance of fluoroscopy. One of such methodologies allows for a sparse electromechanical mapping of the endocardial surface of the left ventricle (LV) by employing a so-called NOGA catheter that is placed into the LV under the X-ray control, which is required because NOGA lacks a capability to provide an anatomical image of the heart. This approach has a number of drawbacks such as exposure to ionizing radiation for both a patient and medical personnel, projection of large three-dimensional (3D) imaging field (through the entire depth of body) onto a two-dimensional (2D) plane, and the necessity to use specialized procedure rooms. In addition, while NOGA allows for detection of the endocardial surface, it cannot detect the motion of the cardiac wall and has limited spatial resolution. Finally, the cost of employing this method and the required stereotactic systems is rather prohibitive.

The described limitations led to a development of a number of new methods for catheter guidance including the use of magnetic navigation, registration of previously acquired images with fluoroscopic and/or ablation system images, electroanatomic voltage-gradient guidance, non-contact mapping systems, and remotely-controlled robotic systems.

One of the methods, which is currently at a stage of experimental proof-of-concept, is an intramyocardial injection catheter tracking with magnetic resonance imaging (MRI) by means of a radiofrequency (RF) antenna with a receiver coil at its tip. This approach was shown to identify an infracted myocardium with the use of real-time MRI for guiding the catheter from a carotid artery. The deficiencies of this not-yet-proven technique include a need in a costly MRI suite, confinement of the catheterization team in proximity to the magnet, and prohibition on use of any metallic instruments.

Another approach, which can be used during the applications of sonomicrometry, is to guide catheter with ultrasound imaging. Sonomicrometry uses, for experimental analyses of local cardiac motion, miniature crystals (typically 1-2 mm in diameter; made, for example, from a piezoelectric material). The crystals transmit to and receive from each other approximately 1-MHz US-pulses at about 250-Hz rate, thereby bringing about a measurement of a distance separating these crystals based on the measurement of the time-of-flight.

While a catheter lends itself to being guided with US during insertion, the obtained US-images suffer from speckle patterns and backscatter pattern ambiguity, easily causing errors in the determination of the position of the catheter tip within the cardiovascular system. Such confusing speckle patterns, SP, are indicated in FIG. 1A showing subendocardial placement of the crystals C1 and C2. FIGS. 1B and 1C illustrate similar limitations of a conventional ultrasound modality in depicting a catheter inside the LV using the ICUS scan and the transthoracic scan, both of which otherwise can be used for basic, approximate guidance of the intervention catheter. It was observed that the simultaneous operation of the sonomicrometer and electrocardiography (echo) suffers from acoustic interference hindering the clarity of US images used for navigation of the sonomicrometric catheter and causing the users to turn off the sonomicrometry system while acquiring echoes and, therefore, causing not saving the sonomicrometric data during imaging.

A skilled artisan shall realize, therefore, that while commonly-available, real-time ultrasonographic systems may satisfy the requirement of being minimally invasive and can be used alone to guide catheters, they have fundamental limitations. The use of ultrasound imaging system alone to guide catheters within human body (and, in particular, within the heart as discussed herein), suffers from a problem of differentiating the actual tip of the catheter from a bend coming in or out of the 2D Doppler plane. Rapidly evolving 3D US imaging is expected to improve spatial determination of objects, including the localization of a catheter. But the transition from 2D to 3D only converts the problem of reliably localizing the tip of the catheter in or out of a 2D plane to a problem of determining the tip location in or out of a 3D Doppler space. The fundamental limitations of ultrasound signal propagation, including refraction, attenuation, rather unpredictable backscatter patterns, and signal drop-outs are sources of imaging artifacts that compromise catheter navigation regardless of spatial dimensionality. Furthermore, the ultrasound image of the catheter tip is often disguised on the background of an image of soft tissue because the backscatter pattern of the catheter is not unique. As a result, a position of the catheter tip is often misinterpreted or determined inaccurately if the actual tip is located out of the scan plane. This could lead to accidental injury or piercing of the cardiac wall.

The present invention overcomes these fundamental limitations of US-imaging in accomplishing minimally-invasive, US-image-guided interventions by employing an active, ultrasonically identifiable catheter tip that can be precisely located in a scan-plane and, furthermore, can be tracked to identify locational deviations of the tip from a scan-plane.

### SUMMARY OF THE INVENTION

According to embodiments of the invention, the acoustic interference that can be often present during the use of US imaging systems (for example, the interference caused by interaction between the signal pulses from an acoustic-signal generating crystalline element and those produced by the US transducer, or the interference between any two acoustic signals produced by transthoracic, transesophageal, intracardiac, and intravascular ultrasound scans) and that is conventionally discarded as strictly unwanted noise, is unexpectedly and advantageously used as an informative interferometric signal for active tracking and navigation of the catheter. More particularly, the present invention provides an acoustically-active catheter (AAC) having a tip that serves as a uniquely identified “beacon” in US-images acquired by the ultrasound imaging system. The system optionally utilizes color-coding of the acquired US-images to facilitate unique identification of the motion of the AAC within the environment of choice, for example within the tissue. Using a principle of interferometric signal-guided navigation of the AAC with the use of PW Doppler ultrasonography, the location of the AAC can be referenced and changed with respect to a given scan plane and to a location of the PW sample within that scan plane with a high degree of accuracy. Consequently, active navigation of the AAC is implemented.

Embodiments of the present invention provide a system for imaging os a bodily system (as a non-limiting example—a cardiovascular system) using ultrasound pulses. The system includes a Doppler ultrasound machine having an ultrasound transducer and a catheter. The latter includes a portion that is distal to the Doppler ultrasound machine and that is configured for insertion into a body and into the cardiovascular system, and a proximal portion operably connected to a controlling mechanism that, in one embodiment, may include hardware and/or software driver such as a function generator and/or a computer program product. In one embodiment, the proximal portion is configured to include a handle of the catheter. The distal portion of the catheter has a steerable tip and a crystalline element affixed at the tip. The ultrasound transducer and the crystal at the tip of the catheter are electrically equipped to generate respectively corresponding acoustic waves that interfere with each other, thereby producing an acoustic interference signal detectable by the system. In a specific embodiment, the electrical equipment of the ultrasound transducer is independent from the electrical equipment of the crystal at the tip of the catheter. The proximal portion of the catheter is equipped with a control mechanism that is movably attached to the distal portion and configured to adjust the location of the active tip of the catheter in response to the acoustic interference signal detected by the Doppler ultrasound machine. In a specific embodiment, the control mechanism is connected to the catheter tip by wiring that is internal to the catheter. The adjustment of the catheter's tip (i.e., spatial tracking of the tip) is carried out in three-dimensional space in such a fashion as to produce changes in intensity of the acoustic interference signal, for example an intensity increase. In a particular embodiment, the spatial tracking is carried out to produce changes in intensity of acoustic interference signal within the US scan plane that projects through the target location. The ultrasound transducer of the system of the invention is configured to operate in a. In addition or alternatively, the US transducer is configured to show the PW sample within the 2D scan plane and may operate in other Doppler modes as well, for example, continuous-wave, color-flow, or power Doppler.

An embodiment of a system is additionally adapted to produce an interference output representing intensity of the acoustic interference signal. In a specific embodiment, such interference output includes a display configured to generate a visual image containing interference fringes and/or an audible signal generated by the system based on the detected acoustic interference signal.

One embodiment of the imaging system additionally includes a tangible computer-readable storage medium containing a computer-program product thereon, where the computer-program product includes program code for at least (i) generating of an image of the catheter tip based on an ultrasound echo produced by the ultrasound waves generated by the transducer and reflected by the tip; (ii) causing a crystalline element at the tip of the catheter to generate an acoustic wave with predetermined acoustic parameters; (iii) determining a location of the tip based on the detection of an acoustic interference signal resulting from interference between an acoustic waves generated by the transducer and that generated by the crystalline element; and (iv) operating a catheter such as to change a position of the catheter's tip in response to the detected acoustic interference signal. The embodiment of the imaging system further contains a processor that is programmable with the above-mentioned program code such as to effectuate active navigation of the steerable tip of the catheter in response to determination of a location of the catheter's tip based on the detection of an acoustic interference and in reference to at least one Doppler scan plane.

Embodiments of the invention also provide a method for tracking and/or controlling of tracking of a catheter, which has a tip equipped with a crystalline element, with the use of an ultrasound imaging system having an ultrasound transducer. Generally, such tracking and/or controlling of tracking is carried out within a bodily system. In one specific embodiment, however, such tracking and/or controlling of tracking is carried out in a cardiovascular system. The crystalline element is adapted to generate acoustic waves. An embodiment of the method includes i) generating an image of the catheter tip arranged within a body based on an ultrasound echo produced by ultrasound waves generated by the transducer and reflected by the tip of the catheter; (ii) detecting an acoustic interference signal formed by a first acoustic wave generated by the transducer and a second acoustic wave generated by the crystalline element; and iii) determining a desired change of a position of the catheter tip in responde ti the acoustic interference signal detected by and transducer. The embodiment may additionally include changing a position of the catheter tip within the body (and, in particular, within the cardiovascular system) in accordance with the results of determining a desired position of the catheter tip.

In a particular embodiment, the first acoustic waves used for producing interference are generated by the transducer operating in a pulsed-wave Doppler mode and the intensity of the acoustic interference signal caused by interference and detected by the system depends on a separation distance between the catheter tip and a Doppler scan plane associated with the transducer.

According to an embodiment of the invention, detecting the acoustic interference signal includes detecting an acoustic interference signal having intensity that depends on a separation distance between the catheter tip and a Doppler scan plane associated with the transducer. Detecting the acoustic interference signal may additionally include generating an output, from the ultrasonic imaging system, that represents an intensity of the acoustic interference signal. In one embodiment, determining a desired change of a position of the catheter tip includes changing a position of the catheter tip in reference to (i) a target within the body (and, in particular, within the cardiovascular system) and (ii) and output that is generated by the ultrasound imaging system in response to the detected acoustic interference signal and that represents a distance between the target and the catheter tip. Changing a position of the catheter tip includes moving the catheter tip so as to increase an intensity of the generated output. In a specific embodiment, the moving of the catheter tip is accomplished in such a fashion as to maximize the intensity of the acoustic interference signal. Consequently, changing a position of the catheter tip includes navigating the catheter tip towards an anatomic target by determining a location associated with maximum intensity of the acoustic interference signal. In a related embodiment, the transducer is operated in a PW Doppler mode and the method additionally includes spatially overlapping a PW Doppler window generated by the transducer with an anatomic target within the body (an, in a specific embodiment, within the cardiovascular system) and generating an output, from the ultrasound imaging system, that represents an intensity of the acoustic interference signal.

Embodiments of the invention also provide for a computer-program product, for use on a computer system for navigating a catheter of an ultrasound imaging system, the computer-program product including a computer-usable non-transitory tangible medium having a computer-readable program code thereon. The computer-readable program code includes program code for causing a crystalline element at a tip of the catheter to generate a first acoustic signal, program code for causing a transducer of the imaging system to generate a second acoustic signal, program code for detecting a signal representing acoustic interference between the first and second acoustic signals, and program code for operating a catheter such as to move its tip into a new position, with respect to a Doppler scan plane formed by the transducer, such as to increase a strength of the signal representing acoustic interference. Alternatively or in addition, the program code includes program code for determining a location of an anatomic target in an environment in which the catheter is disposed, and program code for causing the transducer to generate an acoustic signal in a pulsed-wave Doppler regime such as to spatially overlap the corresponding pulsed-wave Doppler window with this anatomic target.

### BRIEF DESCRIPTION OF THE DRAWINGS

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims herein for interpreting the scope of the invention.

FIGS. 1A, 1B, and 1C demonstrate the operational limitation of methods of the related art, showing multiple imaging artifacts that prevent a reliable direct navigation of the catheter tip. FIG. 1A: an image representing an ultrasound scan of two crystals subendocardially implanted in the anteroapical myocardium. Variable speckle patterns (SP) could be mistaken for the location of the crystal. FIG. 1B: intracardiac ultrasound (ICUS) image, taken from the right ventricle, of the acoustically-active catheter (AAC) that is being advanced from the LV cavity towards the posterior wall. Various imaging artifacts could confound determination of the catheter position. FIG. 1C: a transthoracic scan from the parasternal projection.

FIG. 2 is a block diagram of an example of the ultrasonic imaging system, which employs an embodiment of the present invention.

FIG. 3A is a schematic of a steerable intracardiac injection catheter equipped with an acoustically active tip according to one embodiment of the invention.

FIGS. 3B and 3C are pictures of an exemplary Blazer™ catheter; the catheter is furnished with an acoustically active tip according to an alternative embodiment of the invention. FIG. 3B: a system employing the Blazer™ catheter. FIG. 3C: the tip-end of the catheter of FIG. 3B is shown to be deflectable, at different radii, to ensure a movement of the tip in all three spatial dimensions.

FIGS. 4A and 4B illustrate the piezoelectric crystal of an embodiment of the AAC operating as a localized acoustic transmitter. FIG. 4A is an image of a B-mode scan in-vitro, based on which the crystal cannot be clearly distinguished from the imaging artifacts to be guided by a conventional echo-scan. FIG. 4B is a PW Doppler graph showing, in comparison, two graphs related to the detected interference signals. The display on the left of FIG. 4B (denoted as I) shows no interference signal when the crystal at the AAC-tip is out of the PW Doppler sample window. The display on the right of FIG. 4B (denoted as II) (shows a non-zero interference signal when the crystal at the AAC-tip is located inside the PW sample window or volume.

FIG. 5A is a flow-chart showing an algorithm of navigation of an AAC of the invention with the use of interference ultrasonography.

FIGS. 5B, 5C, 5D, and 5E illustrate the navigation of the AAC in-vivo (in a beating heart). FIG. 5B: a 2D B-mode image representing an in-vivo 2D transthoracic scan of the catheter inside a pig's heart when the AAC-tip is within the PW Doppler sample window. FIG. 5C: a graph corresponding to the image of FIG. 5B and showing, with bright vertical lines, the resulting PW Doppler interference signal that is repeated at the pulse repetition frequency of the signal driving the AAC. FIG. 5D: a 2D B-mode image representing an in-vivo 2D transthoracic scan of the catheter inside a pig's heart when the AAC-tip is outside the PW Doppler sample window still within the 2D-imaging plane. FIG. 5E: a graph corresponding to the image of FIG. 5D and showing a weakened interference signal.

FIG. 6A is a flow chart schematically illustrating a method of the invention to interferometric navigation of the AAC-tip with respect to two mutually intersecting Doppler scan planes.

FIG. 6B is a picture illustrating two mutually intersecting Doppler scan planes formed by a 4D imaging transducer and a path of navigation of the AAC-tip towards and along an intersection axis.

FIG. 6C is a picture schematically showing a biplane Doppler scan arrangement centered on an anatomic target.

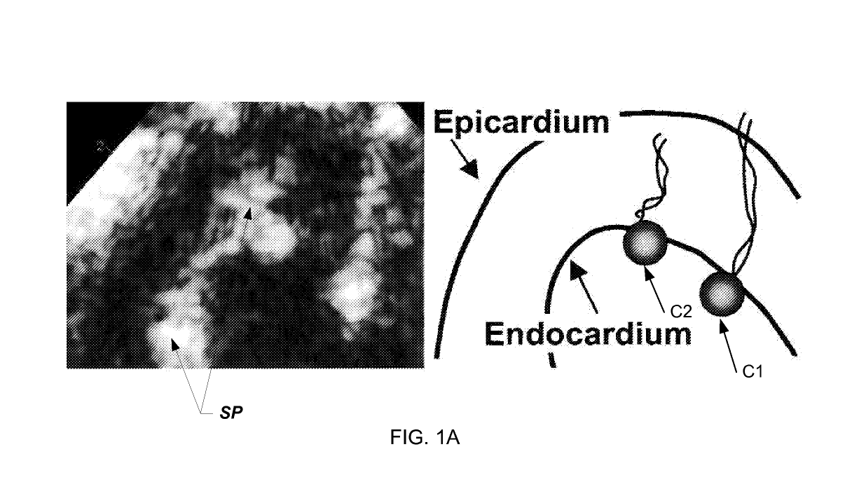

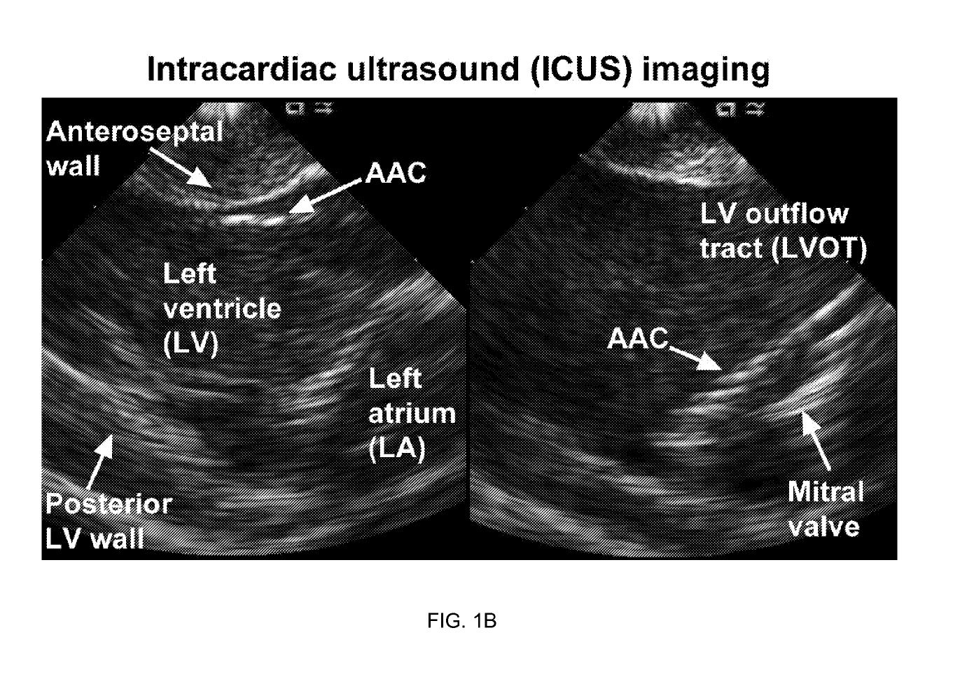

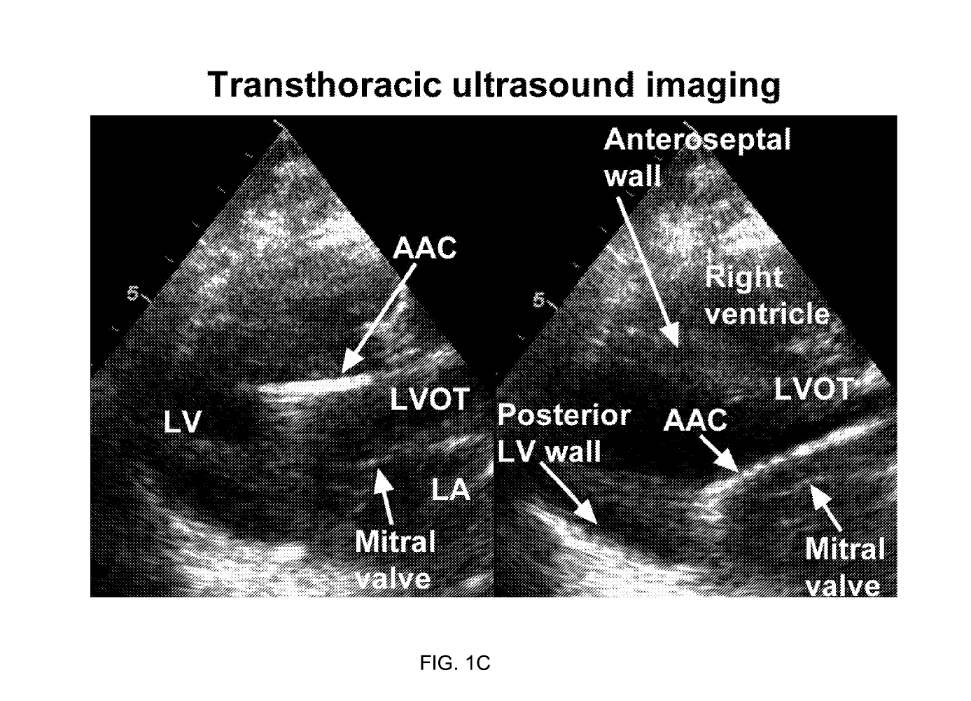

### DETAILED DESCRIPTION OF THE SPECIFIC EMBODIMENTS

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and/or in reference to a figure, is intended to provide a complete description of all features of the invention.

In addition, in drawings, with reference to which the following disclosure may describe features of the invention, like numbers represent the same or similar elements wherever possible. In the drawings, the depicted structural elements are generally not to scale, and certain components are enlarged relative to the other components for purposes of emphasis and understanding. It is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented in this view in order to simplify the given drawing and the discussion, and to direct the discussion to particular elements that are featured in this drawing.

A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily be shown in each and every drawing describing such embodiment, the presence of this detail in the drawing may be implied unless the context of the description requires otherwise. In other instances, well known structures, details, materials, or operations may be not be shown in a given drawing or described in detail to avoid obscuring aspects of an embodiment of the invention that are being discussed.

Furthermore, the described features, structures, or characteristics of the invention may be combined in any suitable manner in one or more embodiments. Moreover, if the schematic flow chart diagram is included, it is generally set forth as a logical flow-chart diagram. As such, the depicted order and labeled steps of the logical flow are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow-chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Without loss of generality, the order in which processing steps or particular methods occur may or may not strictly adhere to the order of the corresponding steps shown.

Consequently, the invention as recited in claims appended to this disclosure is intended to be assessed in light of the disclosure as a whole.

The terms “interference”, “interferometric”, and the like in the context of this disclosure refer to interaction of the signals associated with the crystalline element at the tip of the AAC and the transducer of the US-imaging machine. These signals, referred to interchangeably as “acoustic signals”, “acoustic waves”, and the like, generally have a wide range of acoustic frequencies such, for example, the frequencies in the audible and ultrasound ranges. The ultrasound transducer and US-imaging system may operate in various Doppler modes, for example, pulsed-wave (PW), continuous-wave (CW), and color flow (CF) during identification of the AAC-tip.

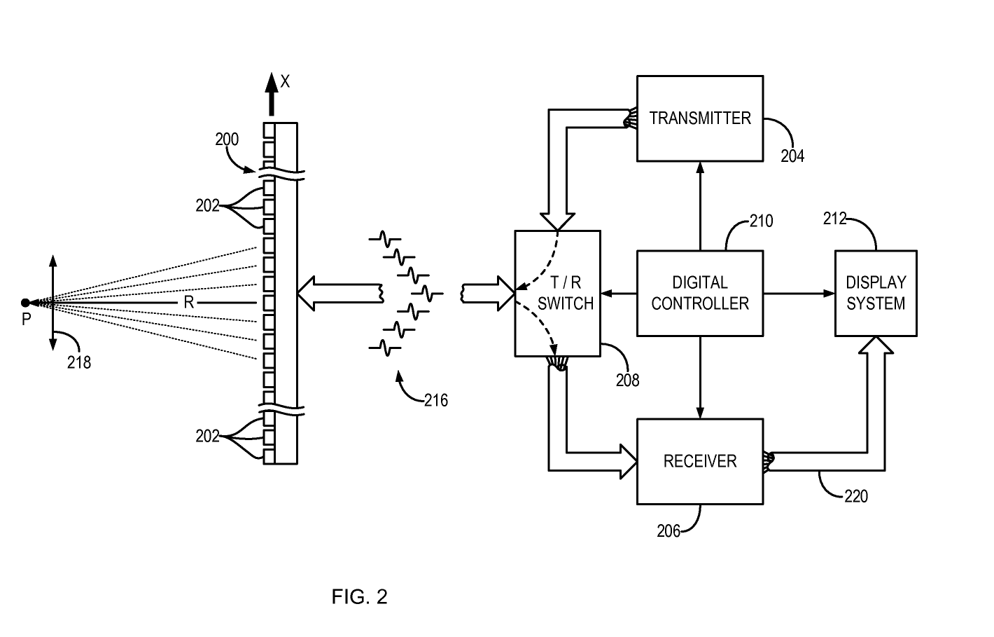

Referring particularly to FIG. 2, an ultrasonic imaging system includes a transducer array 200 comprised of a plurality of separately driven elements 202, each of which produces a burst of ultrasonic energy when energized by a pulse generated by a transmitter 204. The ultrasonic energy reflected back to the transducer array 200 from the subject under study (SUT, not shown) is converted to an electrical signal by each transducer element 202 and applied separately to a receiver 206 through a set of switches 208. The transmitter 204, receiver 206, and the switches 208 are operated under the control of a digital controller 210 responsive to the commands input by a human operator. A complete scan is performed by acquiring a series of echoes in which the switches 208 are set to their “transmit” position, the transmitter 204 is gated “on” momentarily to energize each transducer element 202, the switches 208 are then set to their “receive” position, and the subsequent echo signals produced by each transducer element 202 are applied to the receiver 206. The separate echo signals from each transducer element 202 are combined in the receiver 206 to produce a single “echo” signal that is further employed to produce a line in an image displayed on a display system 212.

The transmitter 204 may drive the transducer array 200 in such a fashion as to direct the produced ultrasonic beam substantially perpendicular to a front surface of the array 200. Referring particularly to FIG. 2, to focus this beam at a range R from the transducer array 200, a subgroup of the elements 202 are energized to produce the beam and the pulsing of the elements 202 that are located in a central portion of the array 200 in this subgroup are delayed relative to those elements 202 that are located in a peripheral portion of the array 200. Consequently, as a result of the interference of the small separate wavelets produced by the subgroup elements, a generated acoustic beam is focused at a point P. The time delay values associated with pulsing of the element 202 determine the depth of focus, or range R, which can be changed during a scan; the purpose of which is to produce a two-dimensional image. The same time delay pattern is used when receiving the echo signals resulting in dynamic focusing of the echo signals received by the subgroup of elements 202. In this manner a single scan line in the image is formed.

To generate the next scan line, the sub-group elements to be energized is shifted by one element-position along the transducer length and another scan line is acquired in a fashion similar to that described above. In operation, therefore, the focal point P of the ultrasonic beam is thus shifted (not shown) along the length of the transducer 200 by repeatedly shifting the location of the energized subgroup of elements 202.

The transducer 200 may be configured to produce an ultrasound beam that is scanned or steered angularly, alternatively or in addition to being scanned along the length of the transducer. For example, in a related embodiment, the transducer 200 is assembled in such a fashion as to have its elements 202 arranged in a two-dimensional matrix, and thereby is configured to produce an US beam that is scanned or steered angularly in two intersecting planes. Addition of such angular steering of the US beam to the longitudinal re-focusing of the beam described above allows the embodiment of FIG. 2 to scan the 3D space.

### Color-Coded Doppler Imaging

Recognition of the motion of an object on a local scale with the use of Doppler US imaging in any number of scans (alternately referred to herein as “image frames”) acquired in a given scan-line is based on correlation between the pulses that are transmitted and reflected along the corresponding scan-lines (or beams), and depth (or distance) from the transducer element 202. A typical two-dimensional (2D) US image frame may consist of hundreds of scan-lines, and a train of US pulses is sent along each scan-line. As a result, formation of each Doppler-image frame requires numerous correlations of pulses to ultimately be displayed on the display system 212.

When an interrogated object is static and does not move, US-pulses reflected from the object in subsequent scans return to the receiver with the same time delay, because the pulses traverse the same round-trip distance between the transducer and the non-moving object. As a result, the corresponding pulses in the subsequent scans are optimally correlated. This optimal correlation indicates to the US-system that the object is not moving along the scan-line with respect to the transducer elements 202.

If, however, the interrogated object is moving, then reflected signals corresponding to two different scans have different time-delays. The time delay associated with a second scan is longer or shorter than that associated with a first scan depending on whether the object is moving away from or towards the transducer, respectively. As a result, there is a change in a degree of correlation between the corresponding pulses in the first and second scans. This change of correlation indicates to the US-system that the object is moving. By electronically “shifting” the pulse obtained in the second scan with respect to the pulse of the first scan, the US-system can be configured, either automatically or with the help of an operator, to find the optimal correlation between the two pulses. The shift needed to recover such optimal correlation is proportional to the displacement of the object along the scan-line that has occurred during the time-delay between the moments when the two subsequent image-frames have been acquired. Since this time-delay is known, the system can calculate both the direction and speed of motion of the object along the scan-line.

As will be described, using these concepts of Doppler imaging, and according to the embodiment of the invention, a local color marker (or overlay) is then associated, in the displayed image and in real-time, with a point along the scan-line at which the pulse reflected from the moving object has been received. The appropriate choice of such color overlay over the image point indicates to the user a direction of motion of the object (for example, red may mean “motion away from” the transducer and blue may mean “motion towards” the transducer, respectively) and speed (expressed, for example, on corresponding red-yellow or blue-green scales or according to any other chosen color-gamut).

In combination with a method of interferometric ultrasonography described below, the use of color-coding offers an operational advantage over the related art in that the proposed technique allows detecting and making visible even stationary objects. The present invention recognizes that, in order to effectuate a detection of the stationary objects, the US imaging system can be configured to interpret a stationary object as a moving object and label or tag it with a color marker representing a “change in position” detected through the interpreted “change in correlation” between the two reflected pulses corresponding to two different scan-lines. Having been appropriately color-coded by the US-system, the tagged image point becomes visible to the operator of the US-system as a colored dot on an image display. Moreover, a specific color-map can be further assigned to the tags so as to differentiate, by color coding, the detected tag-signal from the signals corresponding to ordinary motion of blood-flow, motion of tissue, or motion of navigated object itself (for example, the motion corresponding to advancement of a catheter into the heart).

### Acoustically-Active Catheter (AAC) System and Modes of Operation

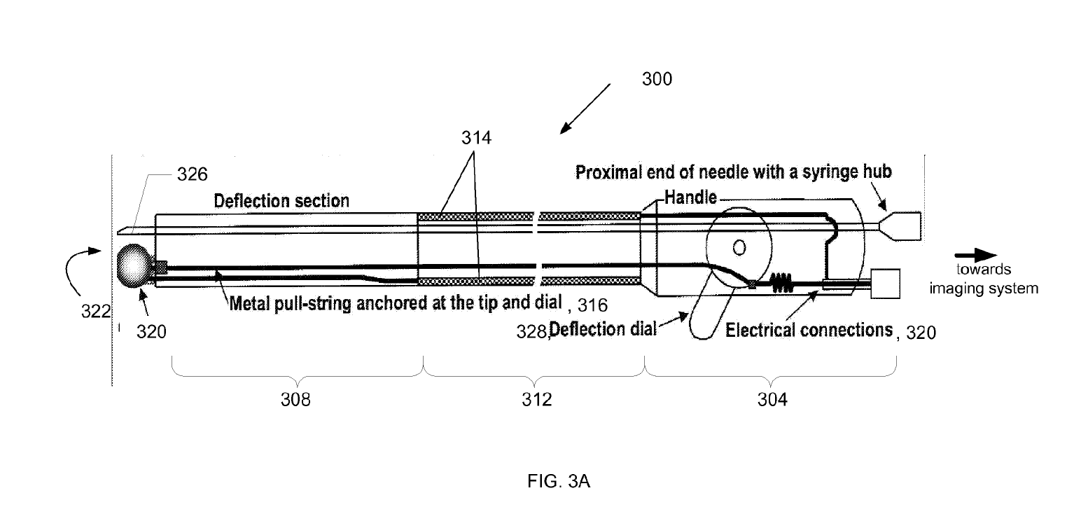

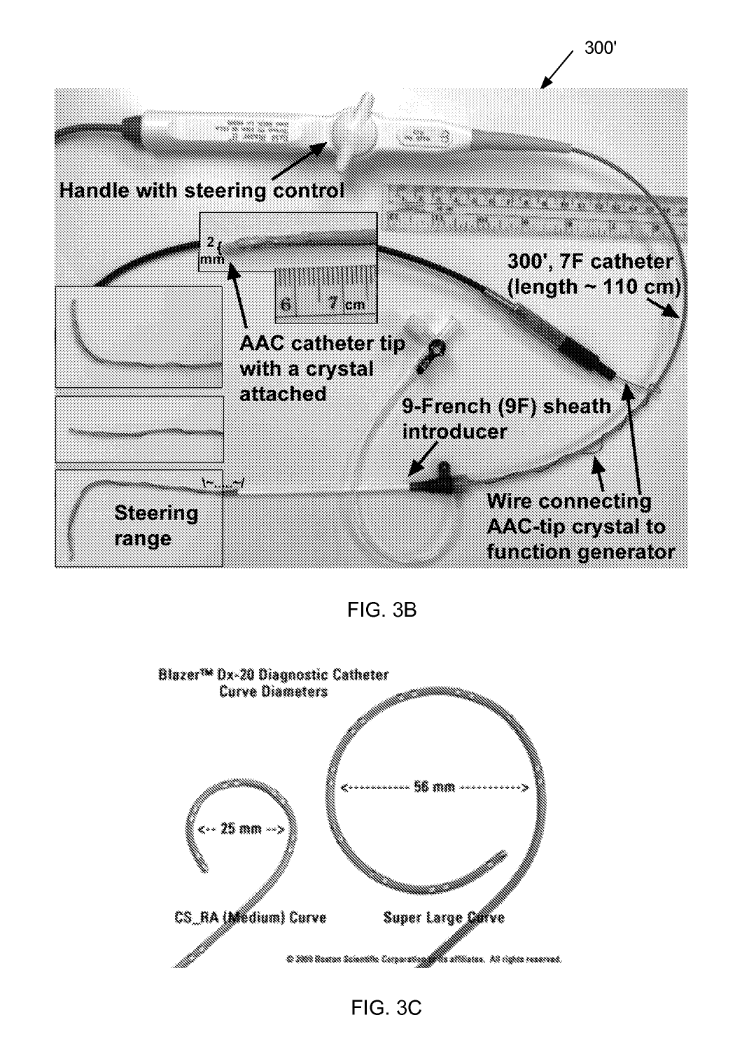

According to one embodiment, at least one miniature piezoelectric crystal is used as an ultrasonic tag in conjunction with a conventional US Doppler system. In particular, a distal end of a steerable catheter is equipped with a small piezoelectric crystal configured to operate in either transmitting or receiving mode, as discussed below, thereby forming an AAC-system of the invention. As schematically shown in FIG. 3A, an exemplary AAC-system 300 may include a proximal end having an AAC-handle 304 and connected to a deflection section 308 of the catheter, which is located at its distal end, through a middle section 312 that may be strengthened with copper braiding 314. A metal pull-string 316 inside the catheter 300 is equipped with a crystalline element 320 (for example, a piezoelectric crystal) that is firmly affixed at the tip 322 of the distal section 308. Both the crystal 320 and the copper braiding 314 are appropriately attached via electrical connectors 324 to the electronic system (not shown) of the embodiment. As shown, the AAC 300 additionally includes a needle 326 and a deflection dial 328 governing the operation of the distal section 308. The AAC-system 300 is further operable with an US-imaging system, as known in the art (not shown).

In a particular implementation, a steerable AAC may employ a commercially-available steering catheter such as a Stiletto or a Myostar catheter. Stiletto is a trademark of Boston Scientific, Inc. (Natick, Mass.) and Myostar is a trademark of Biosense Webster, Inc. (Diamond Bar, Calif.). The Stiletto device, for example, consists of two concentric fixed-curve guide catheters (9 Fr and 7 Fr) and an inner spring-loaded needle component, and the steering of its distal end is achieved by manipulating the positions of the two concentric guide catheters relative to each other. The Myostar device is an 8 Fr deflectable catheter equipped with a 27-gauge extendable and retractable needle having adjustable depth for targeted intramyocardial delivery. The distal tip of the Myostar device is deflected by pulling an internal metal string anchored to the inner side of the distal deflection system. The pull wire simultaneously serves as an electrical connection to the metal tip of the catheter, and the catheter is both strengthened and electrically shielded by copper braiding.

In a related embodiment, a different type if steerable catheter without an injection needle such as a Blazer™ catheter can be used. Blazer is a trademark of Boston Scientific, Inc. (Natick, Mass.). The Blazer catheter has a steerable tip deflectable and is bendable in three dimensions as shown in FIGS. 3(B, C). The deflection movement of the Blazer tip is controlled with a knob in the middle of the handle, which angularly steers the end of the catheter by curving it in a loop shown in FIG. 3C. Control of the rotation of the catheter within a 360 degree angle is provided by twisting the handle, thereby causing the end of the catheter to rotate around the longitudinal axis of the catheter. The length of the distal end of the catheter inserted into the cardiovascular system is controlled by pushing and pulling of the handle at the proximal end of the catheter. A combination of these three degrees of movement—the steering of the tip through angular deflection, the rotational motion of the tip caused by the rotation of the catheter handle, and the controlled longitudinal insertion of the catheter—empowers the user to manipulate the tip of the Blazer™ device within 3D space, such as, for example, an intracardiac chamber.

Besides injection catheters, the AAC-system of the invention can be used with a variety of catheter systems including electrophysiological or ablation catheters and other investigative or surgical tools for catheter guidance during minimally invasive interventions.

According to one embodiment of the invention, the AAC 300 of FIG. 3A or the AAC 300′ of FIGS. 3(B, C) is functionally reconfigurable in that it may be operated as a transmitter or as a receiver. Either type of operation of the AAC can be advantageously used for catheter navigation, as discussed below.

In a transmitting mode, for example, the crystalline element 320 is electrically driven to emit an acoustic signal (a pulse, a train of pulses, or a continuous wave) characteristics of which (such as amplitude, frequency, recurrence) are controllable in reference to the US-system frame rate and/or pulse repetition frequency. Relation(s) between, for example an amplitude (or intensity) and timing of the emitted acoustic signal and the US-system frame rate can be selected to make the catheter reproducibly and uniquely identifiable in US Doppler scans regardless of signal attenuation and ambiguity of backscatter patterns. In a specific embodiment, the crystalline element 320 is configured to transmit in an interferometric regime, when a repetition rate and a frequency of acoustic signal(s) generated by the crystalline element are substantially close to those of the PW Doppler modulation of the US-imaging system with which the AAC is being employed. The choice of this specific regime of operation recognizes that (i) an acoustic interference can be created between the PW Doppler signal generated by the imaging system and the signal emitted by the crystal 320 operating in the interference regime; that (ii) this acoustic interference is more pronounced when the crystal is positioned in proximity to or in a Doppler scan plane; and that (iii) the US-imaging system can detect this acoustic interference and uniquely interpret the resulting interference signal as a spatially-localized representation of the tip of the AAC, thereby distinguishing the AAC on the background of images corresponding to a motion of a blood-flow, a motion of the living tissue, or another background motion produced by the anatomic ROI. According to the invention, the imaging system detects the acoustic interference signal and generates an interference output response to the detected signal. The interference output generated by the system is further adopted by the user to navigate the tip of the AAC to a spatial target, such as that marked with a PW Doppler sample or window, as discussed below. The output response generated by the system may be, for example, an interferometric image displayed on a monitor device and/or an audible signal generated by the system when the system is appropriately equipped with a digital-to-audio converter. In the following discussion interferometric images are primarily used as examples of the output response of the US-imaging system.

The detection and data-processing of acoustic vibrations produced by the crystal of AAC-tip of an embodiment (for example, the crystal 320 of FIG. 3A) can generally be performed as described below. In color-flow Doppler scans, an ensemble of ultrasound pulses can be described by a pulse-repetition frequency (PRF) or the pulse-repetition time interval (T_{PRF}), which is an inverse of the PRF. The pulse-echo spatial impulse response of a single-point scatterer, the temporal response of the transducer of the system, the thermal and electronic noise n(τ, m), and a signal y(τ, m) received by the US system from the m^{th} transmitted pulse have been described by J. A. Jensen in J. Acoust. Soc. Amer., 89:182-90 (1991) in terms of T_{PRF}, the central frequency f₀ of the transducer and the speed of sound, c. It follows from that description that both the envelope and phase of the received Doppler signal are modulate with the instantaneous radial displacement of the single-point scatterer (which can be viewed as a single point source). The AAC-tip can be confidently approximated as such as single-point scatterer.

It is known that a US transducer can be configured to receive echoes from acoustic interfaces formed due to discontinuities in acoustic impedance at various depths along a path of the acoustic signal towards the ROI. The AAC of the invention can also be operated in the receiving mode advantageously used for navigation of the AAC-tip. This embodiment of the invention recognizes that the acoustic field of a flow Doppler scan plane causes the piezoelectric crystal of the AAC to vibrate and produce oscillations representing a highly localized and detectable by the US system signal indicating that the AAC-tip intersected the Doppler scan plane. Accordingly, in one embodiment, the AAC-tip is navigated through the cardiovascular system based on an acoustic signal received by the crystal of the AAC when the AAC tip is placed within the color-flow Doppler ultrasound scan plane.

Interferometric Tracking of the AAC with the Use of Ultrasound Imaging System

A person skilled in the art would appreciate that, due to the interferometric nature of the interaction between an acoustic wave emitted by the piezoelectric crystal at the tip of the AAC and that of the PW Doppler signal of the imaging system transducer, both the intensity of the resulting interference signal detected by the imaging system and the intensity of the corresponding interferometric image displayed by the system to the user depend inversely on the distance between the tip of the AAC and the chosen Doppler scan plane. A movement of the AAC towards the Doppler scan plane, therefore, is accompanied by an increase of the intensity of the corresponding interferometric signal, while a movement of the AAC away from the Doppler scan plane reduces such intensity. The user can then advantageously exploit this dependency to initially navigate the tip of the AAC towards or away from a Doppler scan plane and, once the resulting interferometric image is acquired, towards or away from a PW Doppler sample or window positioned in this Doppler scan plane. While some examples of such navigation are presented below in reference to a cardiovascular system, this particular reference is considered only for the sake of simplicity of explanation and it is understood that, generally, embodiments of the invention are operable within and should be considered with respect to a body and an anatomic target chosen within the body.

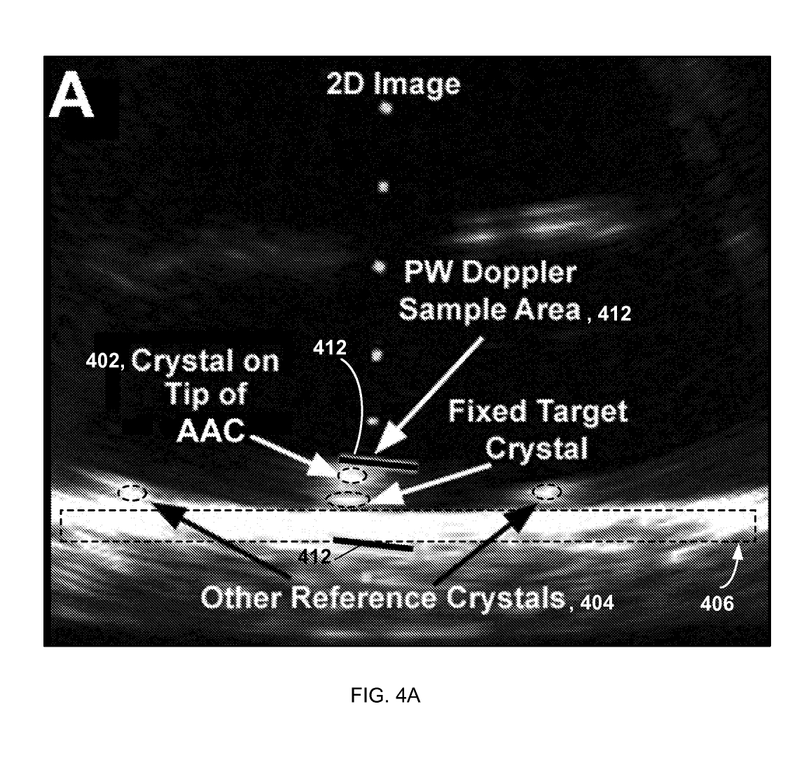

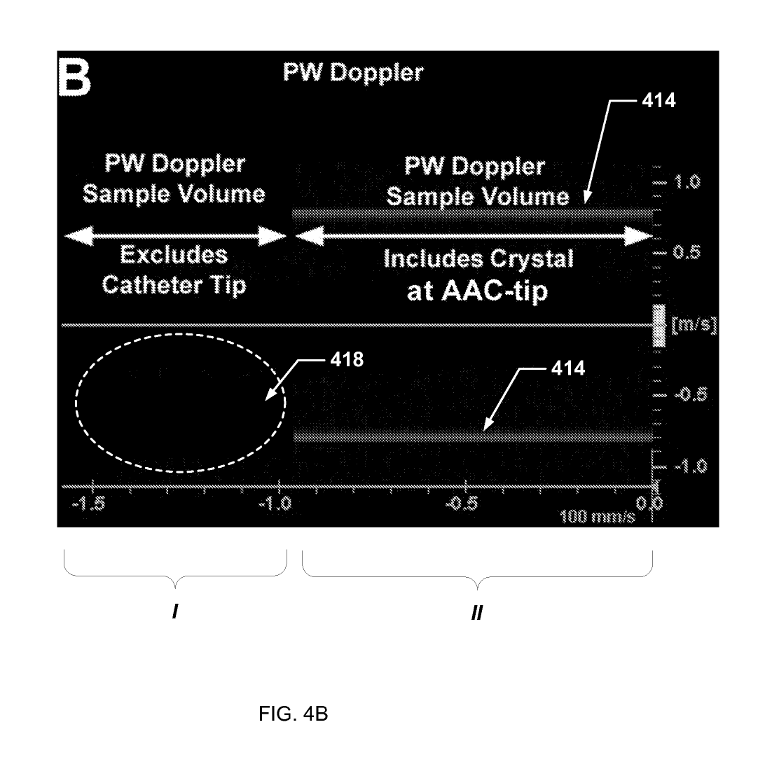

FIGS. 4A and 4B are images illustrating the application of the above-discussed interferometric regime of operation of the AAC to navigation of the AAC-tip to a Doppler scan plane, within a water tank. Here, the ultrasound system is set to a default B-mode scan (1.7 MHz fundamental frequency, 3.4 MHz harmonic frequency) at maximum power. As seen from a conventional in-vitro echo-scan image shown in FIG. 4A and in further reference to FIG. 3A, the tip 322 of the AAC 300, while visually identifiable through its image 402, appears to be substantially similar to images 404 of other piezoelectric crystals and an image 406 of the tissue-density rubber seen at the bottom of FIG. 4A. In FIG. 4A, all of the abovementioned images are outlined with a dashed-line boundary, to facilitate the identification of the images. Overlapped with the image of the B-mode scan of FIG. 4A there is shown a location of a target, a pulsed-wave (PW) Doppler sample 412. The extraneous images 404, 406 produce visual noise that limits the ability of the operator to precisely navigate the tip of the AAC or to visually differentiate it from other portions of the overall image of FIG. 4A. However, having initially identified the presence of the AAC with the conventional echo-scan image such as that of FIG. 4A, the PW Doppler mode of the US-imaging system can be further used, according to a method of the invention, to interferometrically navigate the tip 322 of the AAC 300 with the activated crystal 320 to the chosen Doppler scan plane. A skilled artisan will recognize that such navigation does not require knowledge of the actual position of the catheter with respect to the target. The proposed method of navigation is not tied to the use of fluoroscopy and is not cost-prohibitive because it may be implemented with commercially-available Doppler echo US systems. The process of navigation turns on a determination of the optimal intensity of the interferometric visual output formed by the imaging system (or, in addition or alternatively, on a determination of the optimal intensity of the interferometric audible output produced by an echo machine of the system.)

FIG. 4B illustrates interferometric images produced as a result of interferometric detection of the activated crystal, of the AAC tip, transmitting a continuous sinusoidal signal with a frequency of 2 kHz and an amplitude of 10 volts peak-to-peak. The right-hand portion II of FIG. 4B shows bright images 414 of interferometric signals produced by the system when the transmitting crystal 320 is positioned exactly within a PW Doppler sample 412 of FIG. 4A and, at the same time, within the B-mode Doppler scan plane. The left-hand portion I of FIG. 4B shows the absence, 418, of the signal and associated interferometric fringes when the vibrating crystal 320 of FIG. 3A is positioned several centimeters outside of the PW Doppler sample window 412 of FIG. 4A but still within the plane of the Doppler scan. Similarly, the loss or disappearance of the identifying PW Doppler signal occurs if either the AAC-tip were completely out of the 2D imaging plane or in the plane but not near the PW sampling window.

It is observed, therefore, that in a PW Doppler mode, the difference between the active AAC-tip being within or outside the 2D scan plane is visualized as a detectable change in the strength of the interferometric output produced by the US system. When the crystal at the AAC-tip operates by transmitting a continuous sinusoidal wave, placing the PW Doppler sample window 412 over the AAC-tip uniquely identifies the AAC-tip and distinguishes it from other objects the images 404, 406 of which appear similar to that of the tip in the 2D B-mode image. In this regime, the presence of bright lines 414 on the PW Doppler graph indicates that the AAC-tip is located within the PW sample area window 412.

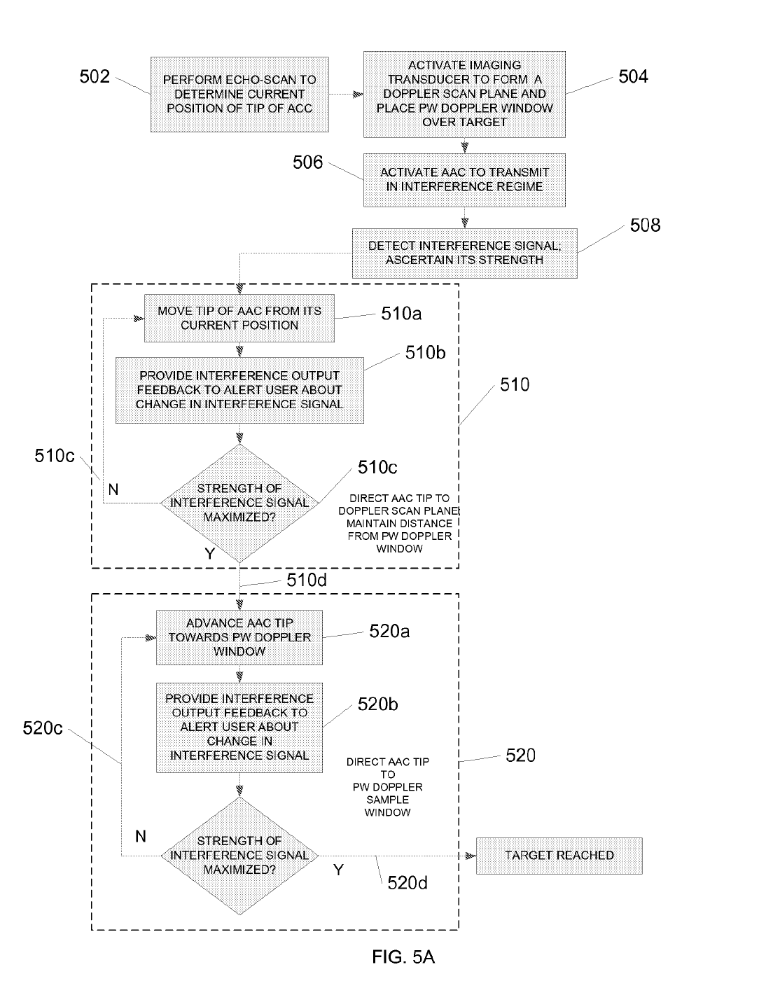

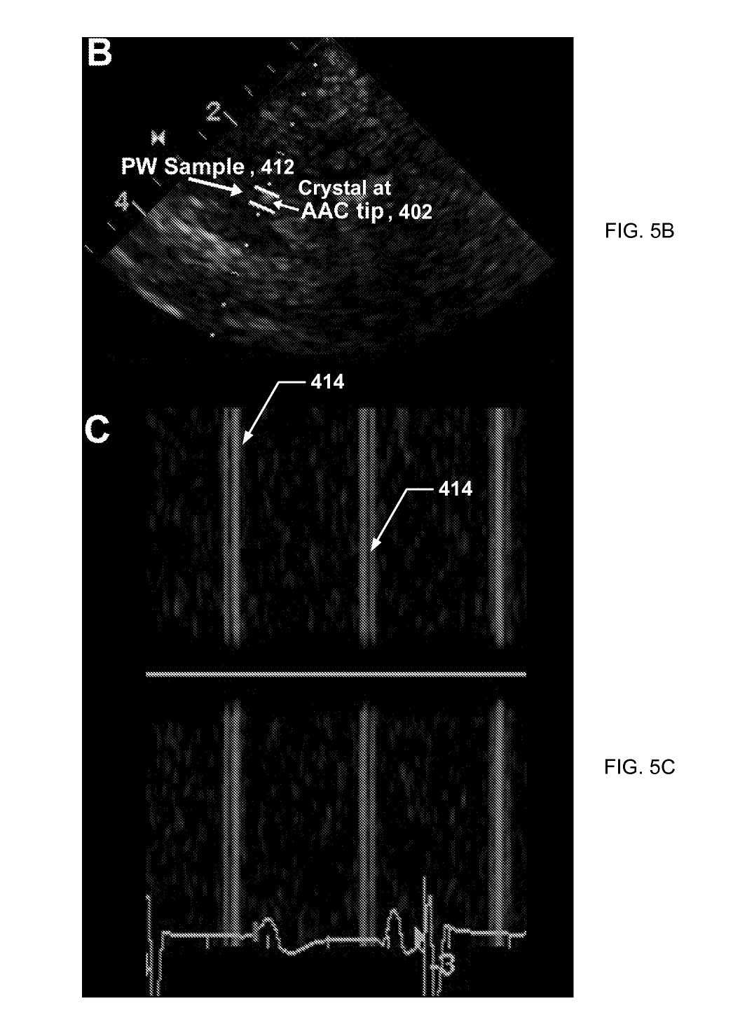

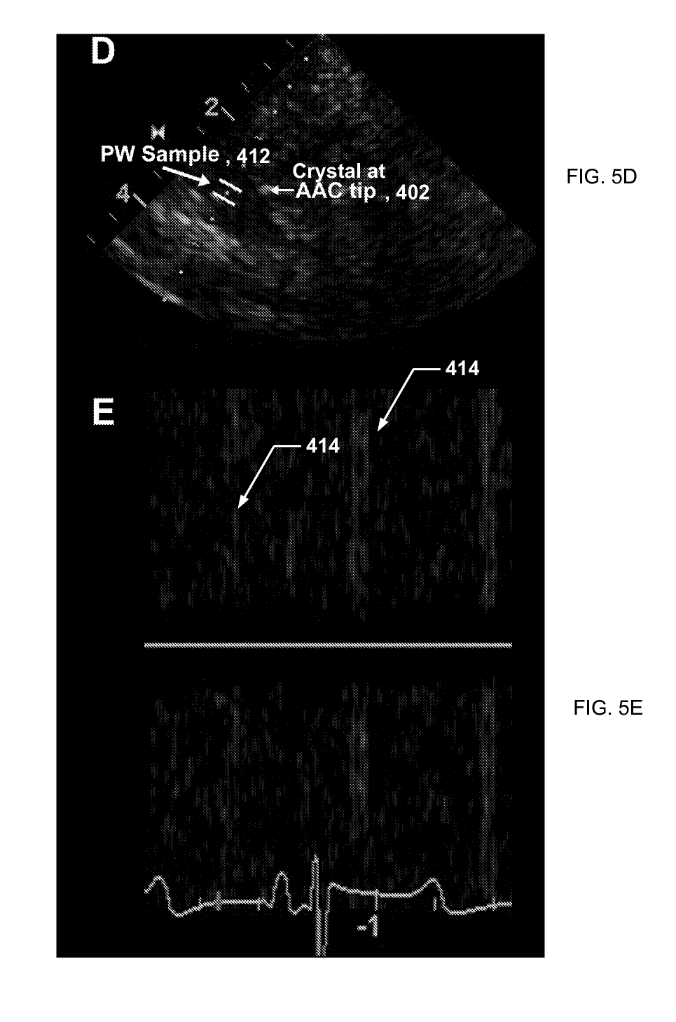

FIG. 5A is a flow-chart of the process of AAC-tip navigation towards a target located in a Doppler scan plane. Following (or, optionally, contemporaneously with) performing a conventional echo-scan, at step 502, in order to determine an approximate position of the AAC within the vascular system and placing, at step 504, a PW Doppler window in an appropriate spatial location with respect to the chosen anatomic target, the piezoelectric crystal of the AAC is activated at step 506 as a transmitter operating in the interference regime. The interference signal resulting from the acoustic interference between the signal transmitted by the catheter of the invention and the PW Doppler signal is detected, at step 508, by the US imaging system that generates at least one interference output indicating the strength of the detected interference signal. The interference output from the US imaging system may be an image displayed to the user and/or an audible output, as described above. Based on the imaging system output, the user manually or with the help of a computer system directs, at step 510, the tip of the catheter towards a Doppler scan plane produced by the system by iteratively changing the current position of the tip of the AAC at sub-step 510a in such a fashion as to assure that the detected interference signal is increasing, sub-steps 510b and 510c. The change of the position of the AAC-tip at this step can be carried out while maintaining the separation distance between the tip of the AAC and the PW Doppler window. When the interference output from the US imaging system indicates, 510d, that the detected interference signal reached its local maximum and, therefore, the AAC-tip has been steered to the Doppler scan plane, the user continues the process of AAC-tip navigation at step 520 by advancing the AAC-tip in the Doppler scan plane towards the PW Doppler window. Similarly to the process of step 510, the advancement of the AAC-tip at step 520 may be performed iteratively, 520a, 520b, 520c, based on the interference output feedback by the imaging system that is indicative of the strength of the detected interference signal. When the US system alerts the user that the detected interference signal reached its maximum intensity, 520d, the goal of the active navigation of the AAC of the invention is achieved. It is understood that conventional echo-scan based observation of the advancement of the AAC-tip can accompany and complement the active navigation of the tip according to the described method.

FIGS. 5B, 5C, 5D, and 5E additionally illustrate an application of the interferometric navigation of the AAC-tip using the method of the invention. FIG. 5B demonstrates a transthoracic US scan (2D B-mode image) of the AAC-tip placed inside a beating porcine heart in situ. As can be seen from the corresponding FIG. 5C, the intensity of the navigation interference signal 414 has substantial intensity and is easily perceived when the tip is steered into the target area represented by the PW sample window 412. In contradistinction, as shown in FIGS. 5D and 5E, when the AAC-tip is outside of the PW sample window 412, the interference signal 414 is weakened, and its brightness is drastically reduced, as shown in FIG. 5E (PW Doppler graph). FIGS. 5D and 5E show the same pig experiment as in FIGS. 5B and 5C but now with the AAC-tip outside the target area. During this experiment, the AAC-tip was being driven by an (actively transmitting) gated rectangular (square-wave) signal with a frequency of 1.3 kHz within each chirp (pulse) and a pulse repetition frequency of 2 Hz. The difference between the orientation of the display of the interference signal in FIG. 5C (vertical orientation) and that of FIG. 4B (horizontal orientation) is due to the difference of the signal driving the AAC, i.e, a gated square wave (FIG. 5C) versus a continuous sinusoidal wave (FIG. 4B). Here, the US imaging system is additionally configured to generate a clicking audible signal output, the amplitude of which depends on the motion of the AAC-tip with respect to the target and allows the user to navigate the AAC-tip exclusively in reliance on the intensity of the audible signal.

The above-discussed principle of the catheter navigation towards a single Doppler scan plane can be appropriately extended, according to the idea of the present invention, to a 3D navigation of the AAC. In one embodiment, for example, the 3D navigation can be implemented by generating two intersecting Doppler scan planes with a transducer imaging the 3D space in a real-time bi-plane mode, in time-sequenced stream of spatial image data. In a specific embodiment, these planes may be mutually orthogonal. For the purposes of this disclosure and accompanying claims, a real-time act performed by a system is understood as an act that is subject to operational deadlines from a given event to the system's response to that event. For example, generation of two Doppler scan planes in real-time is understood to be contemporaneous with the process of catheter navigation, while comparison of data in real-time may be one triggered by the system and executed simultaneously with and without interruption of operation of the system during which such comparison is being performed.

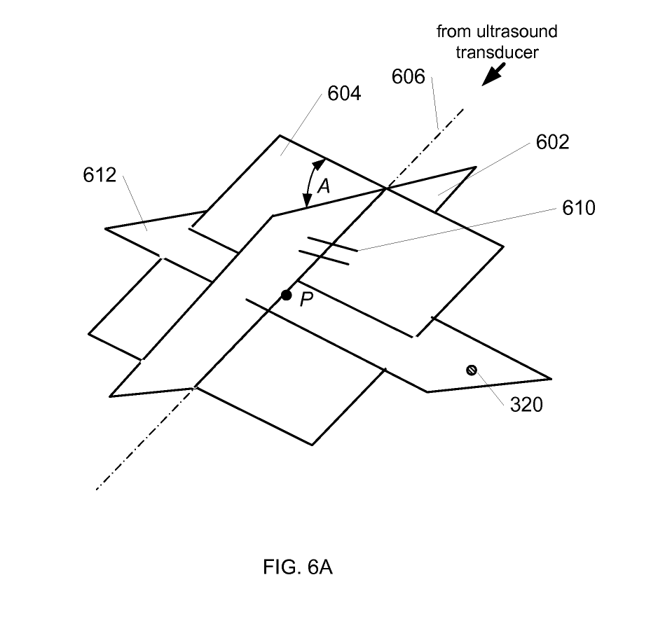

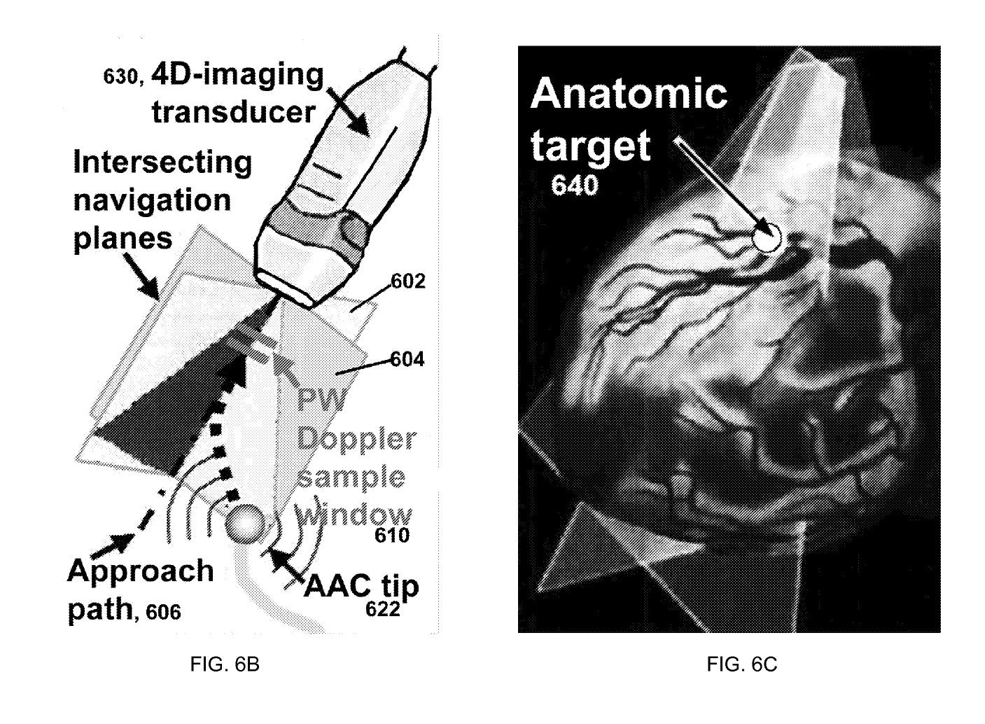

The principle of interferometric navigation of the AAC of the invention in reference to two Doppler scan planes 602 and 604 is further illustrated in FIG. 6A. The Doppler scan planes 602 and 604 are generated by and associated with a transducer (not shown) operating in a 3D-scanning mode. These Doppler scan planes intersect each other along an axis 606 at a dihedral angle A. FIG. 6A also schematically shows the crystalline element 320 that is configured to operate in the interference regime and is affixed to the AAC-tip (not shown). On top of the Doppler scan planes 602, 604 there is shown a PW Doppler sample window 610 that in practice may overlap with and correspond to the position of the chosen anatomic target.

It is appreciated that the virtual axis 606 is a locus of points that are located in both Doppler scan planes 602 and 604. Therefore, the strength of a first acoustic interference signal (that is detected by the US imaging system when the transmitting crystal 320 of the AAC 300 of FIG. 3A is placed at a reference point P on the axis 606) exceeds the strength of any other acoustic interference signal (that is detected when the transmitting crystal is placed at any other point located in a plane 612 through which the axis 606 passes perpendicularly at the reference point P). As a result, the AAC-tip with a crystal 302 (such as the tip 322 of the embodiment 300 of FIG. 3A) transmitting in the interference regime can be actively navigated and the navigation can be controlled (by maximizing the strength of the interference output generated by the US-imaging system as described in reference to FIG. 5A) from its instantaneous location in the plane 612 towards its designated location P on the axis 606. This navigation may include, for example, initially directing the crystal into a first of the Doppler scan planes (such as the plane 602) and then further directing the crystal along that first scan plane towards another Doppler scan plane (such as plane 604), thereby navigating the crystal (and, therefore, the tip of the AAC) along the axis 606 defined by the intersecting planes 602 and 604.

In practice, a 3D interferometric navigation can be used, for example, to pilot the AAC tip to a point at the endocardial surface that is the closest to the epicardial anatomic target. In reference to FIGS. 6B and 6C, the two Doppler scan planes 602, 604 are projected by a 3D-imaging transducer 630 onto an anatomic target 640 such as the coronary artery in the LV cavity. The AAC-tip 322 with the crystal 320 is directed towards the target 640 from inside of the LV along the navigation planes 602, 604 and the intersection axis 606 with a purpose of approaching the target. The determining positions and advancement of the AAC-tip within the cardiovascular system towards the target is indicated by increasing intensity of the “navigation signal” (which is either an interference image or audible interference signal) when the AAC-tip is positioned within one of the Doppler scan planes (602 or 604). By ascertaining the strength of two interference signals respectively corresponding to relative position of the AAC-tip with respect to the planes 602 and 604, the AAC-tip is directed towards the “navigation path” (the axis 606) and is further advanced, as confirmed by a continuously increasing interference signal, towards the PW Doppler sample window that has been appropriately spatially overlapped with a target. In addition or alternatively, the PW Doppler sample window can be positioned in the proximity of the actual anatomic target, for example at the nearest point on the endocardial surface, so as to cause the US imaging system to generate the maximum interference signal prior to an accidental piercing the LV wall with the AAC-tip. As a result, the determination of positions of the catheter tip within the cardiovascular system is carried out in response to the detected acoustic interference signal. The ceasing of the advancement of the AAC-tip during its navigation prior to such accidental piercing of the LV wall can be additionally verified by accounting for the thickness of the LV wall that is simultaneously depicted by and measured with the use of a conventional echo-scan.

While the invention is described through the above-described exemplary embodiments, it will be understood by those of ordinary skill in the art that modifications to, and variations of, the illustrated embodiments may be made without departing from the inventive concepts disclosed herein. For example, although some aspects of a method of the invention have been described with reference to a flowchart, those skilled in the art should readily appreciate that functions, operations, decisions, etc. of all or a portion of each block, or a combination of blocks, of the flowchart may be combined, separated into separate operations or performed in other orders. As another example, a computer-program product containing a computer-readable program code that is configured to effectuate the operation of an embodiment of the above-described system such as to implement the steps of the embodiment of the above-described method are also contemplated to be within the scope of the invention. In addition, although a particular embodiment of the AAC of the invention and a method of its navigation using interference ultrasonography have been described, the disclosed method and structure may be appropriately modified without departure from the scope of the invention. Accordingly, the invention should not be viewed as being limited to the disclosed embodiment(s).

What is claimed is:

1. An apparatus for imaging a cardiovascular system using ultrasound pulses, the apparatus comprising:
a Doppler ultrasound machine having an ultrasound transducer; and
a catheter including
a distal portion having a steerable tip and a crystalline element affixed thereto, wherein the ultrasound transducer and the crystalline element are electrically equipped to generate corresponding first and second acoustic waves adapted to interfere with one another such as to cause an acoustic interference signal that is detectable by the Doppler ultrasound machine; and
a proximal portion having a control mechanism movably attached to said distal portion and configured to adjust a location of the tip in response to said acoustic interference signal detected by the Doppler ultrasound machine.

2. An apparatus according to claim 1, wherein the adjustment of a location of the tip causes a change of intensity of the acoustic interference signal.

3. An apparatus according to claim 1, wherein the ultrasound transducer is configured to operate in a pulsed-wave, continuous-wave, or color Doppler mode.

4. An apparatus according to claim 2, further comprising an interference output configured to indicate an intensity of the acoustic interference signal.

5. An apparatus according to claim 4, wherein the interference output includes a display configured to generate an image containing interference fringes.

6. An apparatus according to claim 4, wherein the interference output is further configured to generate an audible signal based on the acoustic interference.

7. An apparatus according to claim 1, further comprising a controller in operable communication with said proximal portion, the controller being programmed to control an operation of the crystalline element.

8. A method for intravascular tracking of a catheter, having a tip equipped with a crystalline element adapted to generate an acoustic wave, with the use of an ultrasound imaging system having an ultrasound transducer, the method comprising:
generating an image of the catheter tip arranged within a body based on an ultrasound echo produced by ultrasound waves generated by the transducer and reflected by the tip;
detecting an acoustic interference signal formed by a first acoustic wave generated by the transducer and a second acoustic wave generated by the crystalline element; and
determining a desired change of a position of the catheter tip in response to the detected acoustic interference signal.

9. A method according to claim 8, wherein determining a desired change of a position of the catheter tip includes changing a position of the catheter tip so as to increase an intensity of said acoustic interference signal.

10. A method according to claim 8, wherein detecting the acoustic interference signal includes detecting an acoustic interference signal having intensity that depends on a separation distance between the catheter tip and a Doppler scan plane associated with the transducer.

11. A method according to claim 8, wherein determining a desired change of a position of the catheter tip includes changing a position of the catheter tip in reference to a target within the cardiovascular system and an output, which is generated by the ultrasound imaging system in response to the detected acoustic interference signal and which represents a distance between the target and the catheter tip.

12. A method according to claim 8, wherein detecting the acoustic interference signal includes generating an output, from the ultrasonic imaging system, that represents an intensity of the identified acoustic interference signal.

13. A method according to claim 8, wherein generating the image further includes
spatially overlapping a pulsed-wave Doppler window, produced by the transducer, with an anatomic target within the body, and
generating an output, from the ultrasonic imaging system, that represents an intensity of the acoustic interference signal.

14. A method according to claim 13, wherein determining a desired change of a position of the catheter tip includes navigating the catheter tip towards said anatomic target by determining a location associated with maximum intensity of the acoustic interference signal.

15. A method according to claim 13, wherein the catheter tip and the anatomic target are defined within a cardiovascular system of the body.
